(19) United States
(12) Patent Application Publication
(10) Pub. No.: US 2024/0311532 A1
(43) Pub. Date: Sep. 19, 2024

(54) System and Method for Material Modelling and Design Using Differentiable Models

(71) Applicants: Venkatasubramanian VISWANATHAN, Pittsburgh, PA (US); CARNEGIE MELLON UNIVERSITY, Pittsburgh, PA (US)

(72) Inventors: Pin-Wen Guan, Pittsburgh, PA (US); Venkatasubramanian Viswanathan, Pittsburgh, PA (US)

(73) Assignee: CARNEGIE MELLON UNIVERSITY, Pittsburgh, PA (US)

(21) Appl. No.: 18/576,023

(22) PCT Filed: Aug. 22, 2022
(86) PCT No.: PCT/US2022/041009

§ 371 Date: Jan. 2, 2024

Related U.S. Application Data
(60) Provisional application No. 63/239,431, filed on Sep. 1, 2021.

(51) Int. Cl. G06F 30/27

(52) U.S. Cl. CPC G06F 30/27

(57) ABSTRACT

Disclosed herein is a framework for the modeling and design of materials based on differentiable programming, where the models can be trained by gradient-based optimization. Within this framework, all the components are differentiable and can be seamlessly integrated and unified with deep learning. The framework can design and optimize materials for a variety of applications.

### RELATED APPLICATIONS

This application is a national filing under 35 U.S.C. § 371 claiming the benefit and priority to International Patent Application No. PCT/US22/41009, filed Aug. 22, 2022 entitled “System and Method for Material Modelling and Design Using Differentiable Models”, which claims the benefit of U.S. Provisional Patent Application No. 63/239,431, filed Sep. 1, 2021, entitled “Method for Materials Modeling and Design by Differentiable Models”, the contents of which is incorporated herein in their entireties.

### GOVERNMENT INTEREST

This invention was made with United States government support under contract DE-AR0001211 awarded by the U.S. Department of Energy. The United States government has certain rights in the invention.

### BACKGROUND

The three major subjects in materials science are processing, structure and performance. The processing variables (compositions, temperature, pressure, external field, etc.) determine the structure, which further determines the performance. The relation between the processing variables and the phase equilibria (including defects contained in each phase) is described by thermodynamics. The relation between the processing variables and the microstructure (phases, defects and their distribution) can be modeled by the combination of thermodynamic and kinetic models. Although the relation between the structure and some materials properties has been studied in some works using deep learning, for example, the CGCNN (Crystal Graph Convolutional Neural Networks), thermodynamic modeling as the foundation of materials modeling has been previously based on deterministic methodology.

Various phenomena in materials are rooted in the fundamental, well-established laws of thermodynamics. However, thermodynamics of materials is far from being well-studied, due to the vast number of degrees of freedom (i.e., composition, temperature, pressure, strain, external field and dimensionality) and the diverse underlying microscopic mechanisms. In conventional thermodynamic modeling, parameterized models are fitted to collected data of phase equilibria and thermochemistry, but they work for only the chemical systems that pertain to these data and cannot be generalized to the other systems, resulting in low efficiency.

In addition, the underlying microscopic mechanisms contributing to macroscopic thermodynamic observables are diverse, including lattice disorder, atomic vibration, electronic excitation, magnetic excitation, etc., making accurate and comprehensive theoretical descriptions difficult. Therefore, developing a sufficiently efficient methodology is highly crucial for thermodynamic modeling to guide design and synthesis of materials more effectively.

There have emerged many encouraging progresses in recent years. One notable direction is the usage of machine learning (ML) techniques. The generalizability of machine learning allows predictions based on limited amount of data. So far, the methods along this direction can be classified as one or two possible types, based on the types of training data and predicted quantities of the ML models. The type-I models are trained on and predict thermochemical quantities, while the type-II models are trained on and predict phase equilibria. Naturally, one may ask if there can be a cross-type learning, (i.e., learning thermochemical quantities from phase equilibria, or vice-versa).

Machine learning (ML) has the generalizability allowing predictions based on limited amount of data, thus has higher efficiency than model parameter fitting. The primary goal of thermodynamic modeling is learning thermodynamic potentials, which allow calculations of not only phase equilibria, but also useful parameters such as thermodynamic factor in diffusion. However, previous ML techniques can be used to learn thermodynamic potentials from only thermochemical data, due to lacking incorporation of physics linking thermodynamic potentials and phase equilibria.

These types of learning are somewhat similar to the well-known thermodynamic modeling technique CALPHAD (CALculation of PHAse Diagrams). Although the latter lacks machine learning capability, it does have advantages. First, it is often the case that thermochemical data are scarce or not reliable enough, and, as such, training on phase equilibria is the desirable or even the only option. Second, predictions of thermochemical quantities (e.g., Gibbs energies) allows calculations of not only phase equilibria, but also useful parameters such as thermodynamic factor in diffusion. Third, phase equilibria calculated based on thermochemical quantities are more physics-based compared with direct predictions by machine learning.

Therefore, it is of great practical and scientific interest to develop a type of machine learning paradigm for thermodynamics that is able to leverage a broad spectrum of data from high-throughput experiments and first-principles calculations.

### SUMMARY OF THE INVENTION

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

One aspect of the invention comprises a new generalized framework for thermodynamic modeling using the concept of differentiable programming, where all the thermodynamic observables including both thermochemical quantities and phase equilibria can be differentiated with respect to the underlying model parameters, thus allowing the models learned by gradient-based optimization. Thermodynamic modeling and deep learning can be seamlessly integrated and unified within this framework. Thermodynamic modeling in a deep learning style increases the prediction power of models, and provides more effective guidance for design, synthesis and optimization of multi-component materials with complex chemistry via learning various types of data.

Another aspect of the invention comprises a framework for the modeling and design of materials based on the generalized framework for thermodynamic modeling. The framework for the modeling and design of materials comprises one or more inter-related models that can be trained by gradient-based optimization. Within this framework, all the models are differentiable and are seamlessly integrated and unified with deep learning. The framework for modeling and design can design and optimize materials for a variety of applications, for example, lightweight materials, energy storage materials, etc.

### BRIEF DESCRIPTION OF THE DRAWINGS

In the drawings, like reference characters generally refer to the same parts throughout the different views. In the following description, various embodiments of the present invention are described with reference to the following drawings, in which:

FIG. 1 is a block diagram showing a scheme of differentiable thermodynamic modeling.

FIGS. 2(a-d) comprises four graphs showing the evolution of various quantities in the training process for an exemplary model system.

FIG. 3 is a phase diagram of the exemplary model system as predicted by the trained model.

FIG. 4 is a block diagram of one embodiment of a framework for differentiable materials modeling and design.

### DETAILED DESCRIPTION

In one aspect of the invention, disclosed herein is a system and method implementing a new framework capable of learning thermodynamic potentials from both thermochemical data and phase equilibrium data is proposed, wherein thermodynamic modeling and deep learning are seamlessly unified within this framework. This is achieved by introducing differentiable programming (DP), a programming paradigm where the computation flow can be differentiated throughout via automatic differentiation, thus allowing gradient-based optimization of parameters in the flow. The framework learns thermodynamic potentials from both thermochemical data and phase equilibrium data, and seamlessly unifies thermodynamic modeling and deep learning. Due to its ability to incorporate physics into deep learning, DP has increasing applications in different fields, including molecular dynamics, tensor networks, quantum chemistry and density functional theory.

Consider a set of phases {θᵢ}. The thermodynamic potential of the phase θᵢ can be represented as:

$${G}_{{θ}_{i}} ( F ( x ) , C ; {A}_{{θ}_{i}} ) \qquad ( 1 )$$

which is a function of the descriptor F(x) based on composition x, external conditions C (e.g., temperature and pressure) with parameters A. Usage of F(x) is also called feature engineering. When F is an identity (i.e., F(x)=x) raw composition is directly used. G_{θ}_{ᵢ} can be in various forms that are differentiable, such as a conventional one based on polynomials and a more ML-oriented one based on deep neural networks.

Once {G_{θ}_{ᵢ}}, the set of thermodynamic potentials of all the relevant phases are known, each thermochemical quantity gⱼ and phase equilibrium eⱼ can then be calculated, with the calculated values being:

$$= ( \{ {G}_{{θ}_{i}} \} ) , \qquad ( 2 )$$

$$= ( \{ {G}_{{θ}_{i}} \} )$$

which are functionals of {G_{θ}_{ᵢ}}. The loss function can then be computed as:

$$L = {\sum }_{j} l ( {g}_{j} , ) + {\sum }_{j} l ( {e}_{j} , ) \qquad ( 3 )$$

where:
l is a function measuring the difference between two values, with a common choice to be the squared error l(gⱼ, [symbol] )=(gⱼ− [symbol] )².

Finally, the parameter set {A_{θ}_{ᵢ}} of thermodynamic potentials can be obtained by minimizing the loss function:

$$\{ {A}_{{θ}_{i}} \} = arg {min}_{\{ {A}_{{θ}_{i}} \}} L \qquad ( 4 )$$

The minimization of the loss function L relies on calculating its gradient [symbol] L, which is made possible by DP. The most non-trivial part in the above computation flow for differentiation is the phase equilibrium calculation for eⱼ, which generally requires minimization of the thermodynamic potential of the whole system, the major subject of computational thermodynamics with different implementations.

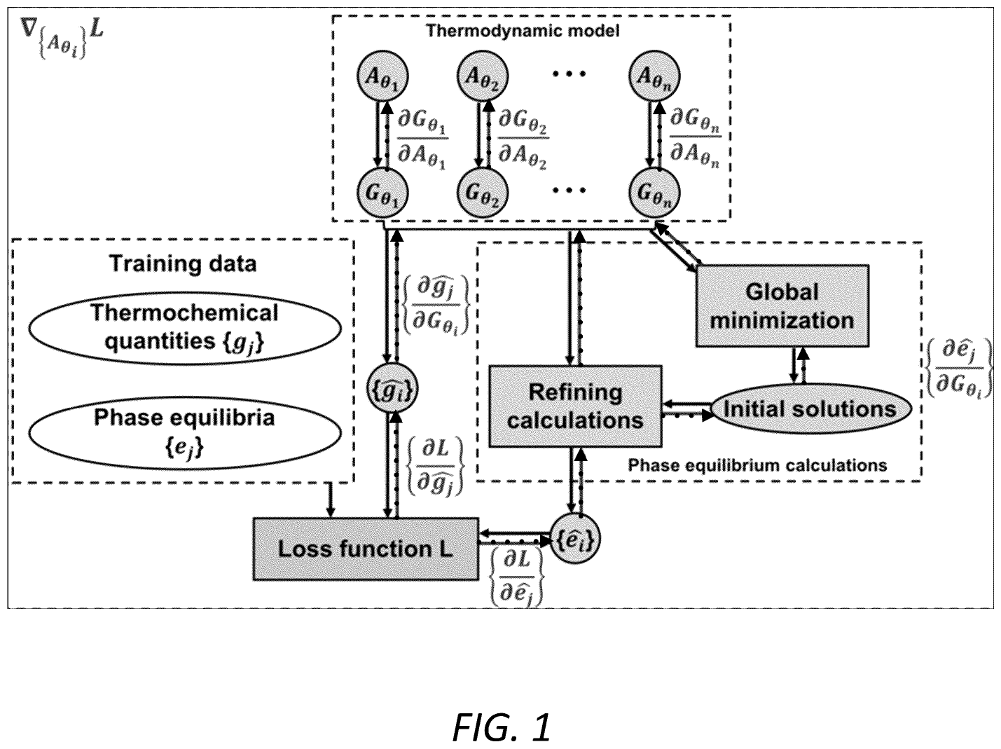

FIG. 1 is a block diagram showing a scheme of differentiable thermodynamic modeling. The forward computation flow is indicated by solid arrows. The backpropagation gradient flow is indicated by dotted arrows, which is used to calculate the gradient of the loss function [symbol] L with respect to model parameters for minimizing the loss function. The variable parts in the differentiation show as filled shapes, while the invariable parts (the training data) have un-filled shapes. The phase equilibrium calculations may vary in different implementations.

The forward mode is similar to routine thermodynamic calculations. For the part of phase equilibrium calculations, the implementation may differ, and here the procedure consisting of a first global minimization step and a successive refining step is tentatively followed. The first step samples the thermodynamic potential surface and generates an initial solution, which is refined to obtain the final solution in the second step. In the backward mode, the computations of gradients propagate from the loss function towards the model parameters through a series of intermediate steps, and all the single steps are finally assembled together to obtain loss gradient [symbol] L based on differentiation. The scheme is similar to that of deep learning, which makes use of multilayer networks and gradient-based optimization to adjust parameters throughout the network based on errors at its output. DP can be regarded as an extension of deep learning with the use of diverse differentiable components beyond classical neural networks.

A general implementation of the above framework that can learn chemical systems with arbitrary complexity requires rewriting or extending a mature computational thermodynamics code to support automatic differentiation. Example

Herein, the purpose is constrained to demonstrate an exemplary thermodynamic modeling framework that supports deep learning formally, rather than applying it to make predictions in a high-dimensional composition/condition space based on large-scale data. For this purpose, a two-phase binary system Cu-Rh is selected. It is sufficiently simple but has some important features in phase diagrams: a liquidus and a solidus between the fcc phase and the liquid (liq) phase besides a fcc miscibility gap right below the solidus. To calculate phase equilibria under given temperature and pressure, Gibbs energy is used as the thermodynamic potential. A four-parameter model is taken as the target to learn:

$${G}_{θ} = {x}_{θ}^{o} {G}_{θ , Rh} + {( 1 - x )}^{o} {G}_{θ , Cu} + RT [ xlnx + ( 1 - x ) ln ( 1 - x ) ] + x ( 1 - x ) ( {A}_{θ} + {A}_{θ}^{′} x ) \qquad ( 5 )$$

where:
θ=fcc, liq;
R is the gas constant;
x_{θ} is the Rh composition in the θ phase; and
°G_{θ,Rh} and °G_{θ,Cu} are the standard Gibbs energies of Rh and Cu in the θ phase, respectively.

The four model parameters have been assessed as (A_{fcc}, A′_{fcc}, A_{liq}, A′_{liq})=(14.609, 11.051, 8.414, 19.799) (in k)/mol) which are regarded as the true values herein the present work. From this set of model parameters, the training data is generated, including phase boundaries at 1000-2200 K.

The Gibbs energy minimization is divided into two steps. The first step is a global minimization on a grid of compositions based on a convex-hull algorithm, generating an approximate solution which is to be refined in the second step. The second step is an iterative self-consistent procedure, where the Newton-Raphson method is used to solve phase equilibria under fixed chemical potential which is then updated based on the newest solved phase equilibria in each iteration, and the iterations stop when convergence is achieved. The loss function for this example system is defined as:

$$L = {\sum }_{T} {L}_{liq - fcc , T} + {\sum }_{{T}^{′}} {L}_{fcc - fcc , {T}^{′}} \qquad ( 6 )$$

$$with :$$

$${L}_{liq - fcc , T} = \{ \begin{matrix}\begin{matrix}α [ {( ( T ) - {x}_{liq} ( T ) )}^{2} + \\ {( ( T ) - {x}_{fcc} ( T ) )}^{2} ]\end{matrix} & if ( T ) \& ( T ) computable \\ {( {min}_{x} \frac{DF}{RT} )}^{2} & otherwise\end{matrix} \qquad ( 7 )$$

$$and$$

$$( 8 )$$

$${L}_{fcc - fcc , {T}^{′}} = \{ \begin{matrix}\begin{matrix}α [ {( ( {T}^{′} ) - {x}_{fcc \# 1} ( {T}^{′} ) )}^{2} + \\ {( ( {T}^{′} ) - {x}_{fcc \# 2} ( {T}^{′} ) )}^{2} ]\end{matrix} & if ( {T}^{′} ) \& ( {T}^{′} ) computa ble \\ {[ ReLU ( \frac{1}{{RT}^{′}} {min}_{x} \frac{{\partial }^{2} {G}_{fcc} ( x , {T}^{′} )}{\partial {x}^{2}} ) ]}^{2} & otherwise\end{matrix}$$

where:
the rectified linear unit ReLU (z)=max(0, z); and
α is a scaling factor for improving convergence in minimization of the loss function.

In the exemplary case, α=100 is used. x_{liq}(T) and x_{fcc}(T) are the liquid composition and the fcc composition in the liquid-fcc equilibrium at temperature T, respectively. x_{fxx#1} (T′) and x_{fxx#2} (T′) are the compositions of the two separated fcc phases in the fcc-fcc equilibrium at temperature T′, respectively. The hats over these phase equilibrium compositions denote the corresponding calculated values. The driving force DF of the metastable phase is the distance in terms of Gibbs energy between the stable tangent plane and a tangent plane parallel to the metastable phase. The loss function in Eq. (6) contains two contributions from the liquid-fcc equilibrium and the fcc miscibility gap respectively. Because the target type of the phase equilibrium may not be correctly reproduced if the model parameters have large deviation from their target values, penalties are imposed instead in such scenarios to favor the regions where

$${min}_{x} DF = 0$$

$$and$$

$${{min}_{x} \frac{{\partial }^{2} {G}_{fcc} ( x , {T}^{′} )}{\partial {x}^{2}} ]}^{2}$$

(i.e., the liquid-fcc equilibrium and the fcc miscibility gap exist at some compositions).

A differentiable program for calculating the above loss function can be written using JAX, a machine learning library which can automatically differentiate Python and NumPy functions. Notably, JAX can differentiate through control flows like loops and branches, which are key structures in the Gibbs energy minimization. The gradient of the loss function [symbol] L is then obtained by automatic differentiation of the program. Given its gradient, the loss function is minimized using a gradient-based optimization algorithm, the L-BFGS-B method.

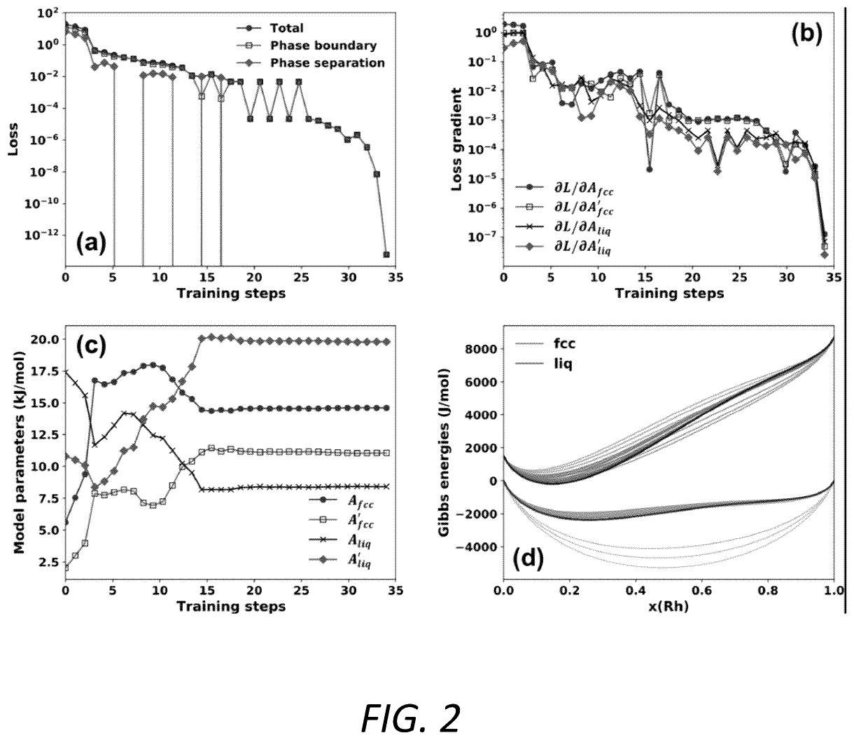

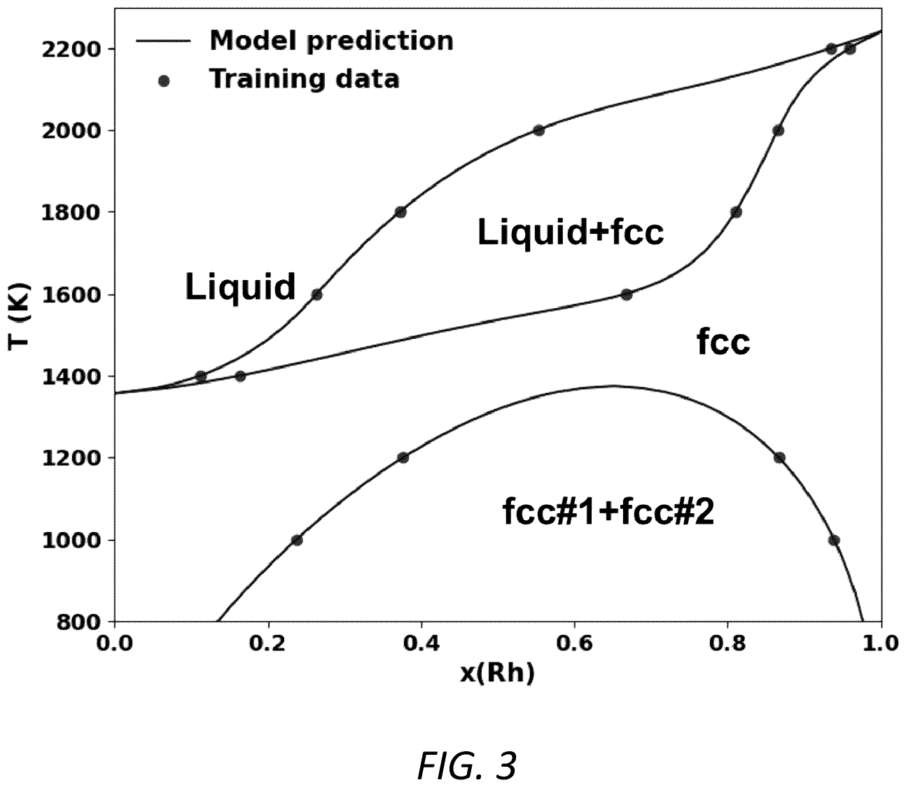

FIGS. 2(a-d) shows four graphs showing the evolution of various quantities in the training process for the Cu-Rh model system: FIG. 2(a) graphs the loss function and the contributions from different origins. FIG. 2(b) graphs the gradient of loss function. FIG. 2(c) graphs the model parameters. FIG. 2(d) graphs the Gibbs energies of involved phases at 1200 K. Heavier colors are corresponded to larger training steps. Despite starting with “unreasonable” initial model parameters, the minimization of the loss function is quite efficient, with good convergence achieved within a few tens of steps, which is made possible by the explicitly calculated gradient of the loss function. In this case, the liquid-fcc equilibrium always exists during training, thus the loss function has only two non-zero contributions from displacement of the phase boundaries (liquid-fcc and fcc-fcc) and absence of the phase separation (fcc miscibility gap) caused by inaccurate model parameters, respectively. The latter contribution vanishes after the fcc miscibility gap is made exist, and then the training is accompanied only with quantitative adjustment of the phase boundaries. With the loss function minimized, its four-component gradient driving the training process approaches the zero vector, and the model parameters also converge to the true values. To better understand how the thermodynamic model evolves, the trajectories of Gibbs energies of the two involved phases at 1200 K in the training are plotted in FIG. 2(d). Consistent with initial absence of the fcc miscibility gap, the Gibbs energy of the fcc phase is initially a convex function without spinodal decomposition, but gradually trained to be non-convex leading to phase separation. The phase diagram of the Cu-Rh system predicted by the trained model is shown in FIG. 3, along with the training data. As can be seen, the predicted phase diagram is in excellent agreement with the training data, meaning the present model training is highly successful.

The above example provides an exemplary preliminary successful application of differentiable thermodynamic modeling, which is able to learn thermodynamics from mixed types of data on thermochemistry and phase equilibria in a deep learning style. Due to simplicity of the binary system and the limited amount of training data, a polynomial with raw elemental compositions directly used as inputs is a suitable form to represent the excess Gibbs energy of each phase, which is also the routine practice in conventional thermodynamic modeling. However, such representation may suffer the “curse of dimensionality” in high-dimension chemical space. For instance, considering an extreme case where a phase contains 100 elements, there are 99 independent compositional variables, C₁₀₀²=4950 binary interactions, C₁₀₀³=161,700 ternary interactions and C₁₀₀⁴=3,921,225 quaternary interaction, totaling a daunting number of model parameters with each interaction represented by a conventional parameterized polynomial. It is therefore desirable to explore an efficient representation of the Gibbs energy or its parameters. Due to its strong expressivity, the neural network is a promising tool for this purpose. Using elemental compositions as inputs, a deep neural network trained on DFT data can achieve a mean average error of 0.05 eV/atom in predicting formation enthalpies. Thus, the neural network can be used to train the models of Gibbs energies efficiently and accurately, and the presently disclosed framework of differentiable thermodynamic modeling can provide a necessary platform for introducing neural networks to learn thermodynamics from diverse types of data.

From the perspective of mapping, a set of phase equilibrium data is actually a sample from the map f: (x, C)=→e, where e is the phase equilibrium at composition x and external condition C. To incorporate more physics, thermodynamic potentials {G_{θ}} is introduced as intermediate variables with two maps f₁: (x, C)=→{G_{θ}} and f₂: {G_{θ}}=→e, which are usually called “thermodynamic model” and “phase equilibrium calculation”, respectively. The map f is just their composition:

$$f = {f}_{2}^{o} {f}_{1} \qquad ( 9 )$$

Note that f₁ and f₂ are very different in nature. f₁ is usually complicated and sometimes even obscure, packaging the whole physics of each single-phase material that is difficult to calculate explicitly without capturing all the atomic and electronic details, but this is just the part in which deep learning can find its largest use. In contrast, f₂ is more straightforward, thus most suitable for a direct physical computation. Differentiable thermodynamic modeling offers a seamless integration of these two components.

The presently disclosed invention comprises a deep learning framework for thermodynamic modeling, which is termed differentiable thermodynamic modeling. It is based on differentiable programming, which allows differentiation throughout the computation flow and therefore gradient-based optimization of parameters. Under this framework, thermodynamics can be learned from different types of data on thermochemistry and phase equilibria in a deep learning style, and thermodynamic modeling and deep learning are de facto unified and indistinguishable. Differentiable thermodynamic modeling can facilitate exploration of thermodynamics of multi-component systems with complex chemistry, as well as design, synthesis and optimization of multi-component materials.

The present invention also comprises a second aspect in which a specific use of the just described general framework using differentiable programming for the modeling and design of materials is disclosed. The framework learns thermodynamic potentials from both thermochemical data and phase equilibrium data, and seamlessly unifies thermodynamic modeling and deep learning. In addition, the general framework has been expanded herein to include additional aspects that can be learned, including, for example, interfacial properties, kinetics, thermochemical quantities, phase equilibria microstructure and performance.

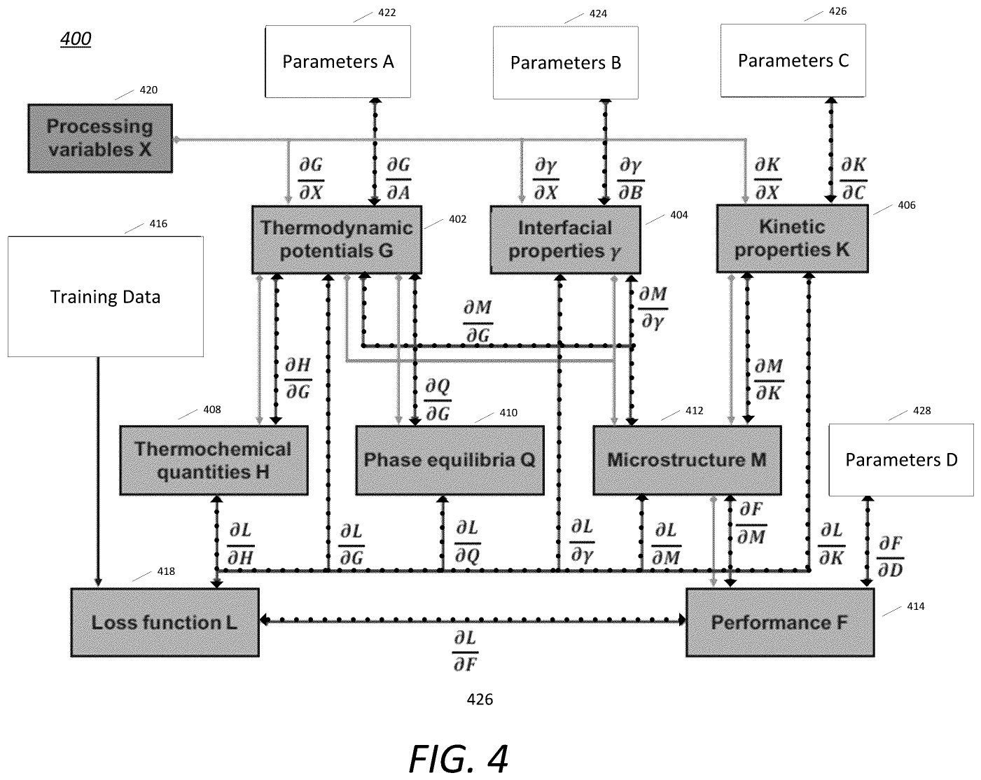

FIG. 4 shows a block diagram of one embodiment of the framework for differentiable materials modeling and design. The dotted and solid double-headed arrows correspond to the modeling and design modes, respectively. The backpropagation gradient flow generally moves from bottom to the top of the figure to the bottom and from the right of the figure to the left and is used to calculate the gradient of the loss function or the performance with respect to model parameters. The forward computation flow generally moves from the top of the figure to the bottom and from the left of the figure to the right. The single-headed arrow between the “Training Data” and the “Loss function L” boxes represents the contributions of the training data to the loss function, which is considered fixed when the training data is given. Each box filled with the [purple color] represents not only a quantity, but also a differentiable function or calculator for deriving this quantity.

In some embodiments, the invention described herein includes one or more of the following components: a differentiable thermodynamic model describing the thermodynamic potentials of relevant phases and/or defects (Thermodynamic Potentials 402), a differentiable model describing the interfacial properties between relevant phases (Interfacial Properties γ 404), a differentiable model describing the kinetic properties of relevant phases (Kinetic Properties K 406); a differentiable calculator for thermochemical quantities (Thermochemical Quantities 408), a differentiable calculator for equilibrium phases and/or defects (Phase Equilibria 410), a differentiable calculator for microstructure (Microstructure M 412); a differentiable calculator for performance (Performance F 414); a database containing training data from experiments and first-principles calculations (Training Data 416); and a differentiable loss function (Loss Function L 418).

The relation of the various components in the framework of differentiable materials modeling and design 400 is shown in FIG. 4. Not only is each component itself differentiable, but also these components are differentially connected, forming a differentiable network, allowing an end-to-end training based on the whole or part of the network. In FIG. 4, each of boxes 402, 404, 406, 408, 410 and 412 represents both the property being calculated and the calculator for calculation of the property. The ML capability of the framework is also related to the usage of the parameterized functions representing Thermodynamic Potentials G 402, Interfacial Properties γ 404, Kinetic Properties K 406, and Performance F 414. Examples of parameterized functions include elementary functions, neural networks, etc.

In some embodiments, Thermodynamic Potentials G 402 is a set of parameterized functions with each function representing the thermodynamic potential of a phase. When the phase has fixed composition, the function is a parameterized function of processing variables except composition. When the phase has variable composition, the function is the sum of composition-weighted terms, a term based on ideal mixing entropy and an excess term describing non-ideal mixing behavior, and each of these terms is a parameterized function of processing variables including composition.

In some embodiments, Interfacial Properties γ 404 is a set of parameterized functions with each function representing a property of the interface between two different or same phases among all the relevant phases. The function is a parameterized function of processing variables and crystal orientation and termination. Examples of interfacial properties include interfacial energy, grain boundary energy, surface energy, interface-defect interaction, interface-solute interaction, etc.

In some embodiments, Kinetic Properties K 406 is a set of parameterized functions with each one representing a kinetic property of a phase. When the phase has fixed composition, the function is a parameterized function of processing variables except composition. When the phase has variable composition, the function is the sum of composition-weighted terms and an excess term describing mixing behavior, and each of these terms is a parameterized function of processing variables including composition. Examples of kinetic properties include diffusion coefficient, kinetic barrier, etc.

In some embodiments, Thermochemical Quantities H 408 is a calculator supporting automatic differentiation. Its input is a thermodynamic potential, and its output is thermochemical quantities such as enthalpy, entropy, heat capacity, etc.

In some embodiments, Phase Equilibria Q 410 is a calculator supporting automatic differentiation. Its input is thermodynamic potential, and its output is types and fractions/concentrations of equilibrium phases and/or defects, as well as chemical composition of each equilibrium phase. The equilibrium here can be a global equilibrium involving all possible phases or a constrained equilibrium excluding some phases.

In some embodiments, Microstructure M 412 is a calculator supporting automatic differentiation. Its input is thermodynamic potential, interfacial properties and kinetic properties, and its output is microstructure (i.e., types, fractions/concentrations, and distribution of phases and/or defects) or descriptors for microstructure. An example of Microstructure M 412 is a differentiable phase field simulator. The term “calculator” here has a generalized meaning, which can be also a neural network or any other function or functionals.

In some embodiments, Performance F 414 is a calculator supporting automatic differentiation. Its input is microstructure or descriptors for microstructure, and its output is performance, which can be a metric based on one or more properties with structural and/or functional applications. An example of Performance F 414 is a differentiable FEM simulator that can calculate mechanical properties. The term “calculator” here has a generalized meaning, which can be also a neural network or any other function or functionals.

In some embodiments, the Training Data 416 can be obtained from experiments and/or first-principles calculations, and can be data about thermodynamic potentials, interfacial properties, kinetic properties, thermochemical quantities, phase equilibria, microstructure or performance.

In some embodiments, Loss Function L 418 is a loss function supporting automatic differentiation. Its input is Training Data 416 and the corresponding model predictions, and its output measures disagreement between Training Data 416 and model predictions.

In some embodiments, Processing Variables X 420 is a set of variables that define the processing conditions for synthesizing the material, for example, temperature, pressure, chemical composition, external fields (e.g., electric, magnetic), etc.

In some embodiments, parameters A 422, Parameters B 424, Parameters C 426 and Parameters D 428 are the parameters needed to establish relationships between an input and a target property. For example, Thermodynamic Potentials G 402 have the processing variables X as the input, so Parameters A 422 represents the parameter set needed to establish the relationship between X and G. For example, suppose G=(G₁, G₂), X=(X₁, X₂) and G₁=2*X₁+5* X₂, G₂=3* X₁−4*X₂, then A can be written as A=[(2,5), (3−-4)]. Similarly, Parameters B 424 represents the parameter set needed to establish the relationship between X and Interfacial Properties γ 404, Parameters C 426 represents the parameter set needed to establish the relationship between X and Kinetic Properties K 406, and Parameters D 428 represents the parameter set needed to establish the relationship between Microstructure M 412 and Performance F 414.

In some embodiments, the differentiable materials modeling and design framework 400 has two modes, a modeling mode and a design mode. In modeling mode, Loss Function L 418 is minimized to train Parameters A 422, B 424, C 426 and D 428 associated with Thermodynamic Potentials G 402, Interfacial Properties γ 404, Kinetic Properties K 406 and Performance F 414, respectively (i.e., finding the parameters using the loss function). In design mode, Performance F 414 is maximized to obtain the optimal Processing Variables X 420. A typical scenario of materials design is that, initially the relationship between Processing Variables X 420 and the Performance F 414 is not completely known, so modeling is needed. After the relationship is known, the design mode is used to select proper Processing Variables X 420 to optimize the Performance F 414.

In some embodiments, the parameters in the differentiable materials modeling framework 400 can be trained together or separately. If only the parameters associated with Thermodynamic Potentials 402 are trained, the modeling is reduced to differentiable thermodynamic modeling.

As would be realized by one of skill in the art, the disclosed systems and methods described herein can be implemented by a system comprising a processor and memory, storing software that, when executed by the processor, performs the functions comprising the method. For example, the training, testing and deployment of the model can be implemented by software executing on a processor.

Further, the invention has been described in the context of specific embodiments, which are intended only as exemplars of the invention. As would be realized, many variations of the described embodiments are possible. The invention is not meant to be limited to the particular exemplary model disclosed herein. Moreover, it is to be understood that the features of the various embodiments described herein were not mutually exclusive and can exist in various combinations and permutations, even if such combinations or permutations were not made express herein, without departing from the spirit and scope of the invention. Accordingly, the method and apparatus disclosed herein are not to be taken as limitations on the invention but as an illustration thereof. For example, as would be realized by one of skill in the art, the differentiable materials modeling framework 400 can be implemented by all or any combination of the modules described above or can include other modules not described herein. Further, variations of embodiments of the differentiable materials modeling framework 400, including variations of both software and hardware components, are still be considered to be within the scope of the invention, which is defined by the following claims.

1. A system for the modeling and design of materials comprising:
one or more interrelated differentiable models for predicting one or more thermodynamic properties of a material;
wherein the thermodynamic properties for a particular phase of the material is a function of a composition of the material and external conditions, given a set of parameters to be optimized by minimizing a loss function constructed from the one or more interrelated differentiable models; and
wherein the one or more differentiable models comprises a differentiable thermodynamic potentials model describing the thermodynamic potentials of relevant phases and/or defects of the material.

2. The system of claim 1 wherein the one or more differentiable models are learned from thermodynamic potentials from both thermochemical data and phase equilibrium data.

3. The system of claim 2 wherein the set of parameters are obtained by minimizing a loss function constructed from the one or more differentiable models.

4. The system of claim 3 wherein the loss function is minimized by iteratively applying a loss gradient.

5. The system of claim 1 wherein the thermodynamic potentials model comprises set of parameterized functions with each function representing the thermodynamic potential of a phase or a defect of the material.

6. The system of claim 1 wherein the one or more differentiable models further comprises a differentiable interfacial properties model describing interfacial properties between relevant phases of the material.

7. The system of claim 6 wherein the interfacial properties model comprises a set of parameterized functions with each function representing a property of an interface between two different or same phases among all the relevant phases of the material.

8. The system of claim 6 wherein the one or more differentiable models further comprises a differentiable kinetic properties model describing the kinetic properties of relevant phases of the material.

9. The system of claim 8 wherein the kinetic properties model comprises a set of parameterized functions with each one representing a kinetic property of a phase.

10. The system of claim 8 wherein the one or more differentiable models further comprises a differentiable calculator for thermochemical quantities of the material.

11. The system of claim 10 wherein the calculator for thermochemical quantities takes as input thermodynamic potentials from the thermodynamic potentials model, and outputs one or more thermochemical quantities of the material.

12. The system of claim 10 wherein the one or more differentiable models further comprises a differentiable calculator for equilibrium phases and/or defects of the material.

13. The system of claim 12 wherein the calculator for equilibrium phase takes as input thermodynamic potentials from the thermodynamic potentials model and outputs types and fractions/concentrations of equilibrium phases and/or defects of the material, and chemical composition of each equilibrium phase.

14. The system of claim 12 wherein the one or more differentiable models further comprises a differentiable calculator for microstructure of the material.

15. The system of claim 14 wherein the calculator for microstructure takes as input thermodynamic potentials from the thermodynamic potentials model, interfacial properties from the interfacial properties model and kinetic properties from the kinetic properties model and outputs a microstructure of the material or descriptors for microstructure of the material.

16. The system of claim 14 wherein the one or more differentiable models further comprises a differentiable calculator for performance of the material.

17. The system of claim 16 wherein the calculator for performance takes as input microstructure from the calculator for microstructure and outputs performance.

18. The system of claim 17 wherein performance is a metric based on one or more properties with structural and/or functional applications.

19. The system of claim 10 wherein the loss function is differentiable.

20. The system of claim 19 wherein the loss function takes as input training data and the predictions from the one or more differentiable models and outputs a quantity that measures disagreement between training data and model predictions and further wherein the loss gradient is derived by differentiating the loss function.

21. The system of claim 1 further comprising a database of training data.

22. The system of claim 21 wherein the training data is obtained from experiments and/or calculations and comprises one or more of thermodynamic potentials, interfacial properties, kinetic properties, thermochemical quantities, phase equilibria, microstructure and performance, and any other related quantities.

23. The system of claim 21 wherein the system operates in either a modeling mode or a design mode.

24. The system of claim 23 wherein, in modeling mode, the system minimizes the loss function to train all or part of the parameters associated with the thermodynamic potentials model, the interfacial properties model, the kinetic properties model and the calculator for performance.

25. The system of claim 23 wherein, in design mode, the system selects a set of processing variables to optimize performances.

26. The system of claim 25 wherein the set of processing variables comprises one or more of temperature, pressure, chemical composition, size, shape, strain of the material and external stimuli such as electric field and magnetic field.

27. The system of claim 1 wherein the one or more models are differentially connected to each other to form a differentiable network.

28. The system of claim 11 wherein the thermodynamic potentials model, the interfacial properties model, the kinetic properties model and the performance calculator take parameters or parameterized functions as input.

29. The system of claim 20 further comprising:
a processor; and
software that, when executed by the processor, implements the one or more interrelated differentiable models and operates the system in either modeling mode or design mode.

30. The system of claim 1 wherein the one or more interrelated differentiable models includes one or more of a thermodynamic potentials model, an interfacial properties model, a kinetic properties model and a performance calculator and further wherein the one or more interrelated differentiable models are based on machine learning wherein the training of the one or more interrelated differentiable models is a machine learning process.
